(19) United States
(12) Patent Application Publication
(10) Pub. No.: US 2025/0262433 A1
(43) Pub. Date: Aug. 21, 2025

(54) NONINVASIVE ELECTRICAL STIMULATION METHOD TO FACILITATE EVALUATION AND TREATMENT OF DYSPHAGIA

(71) Applicant: THE CURATORS OF THE UNIVERSITY OF MISSOURI, Columbia, MO (US)

(72) Inventors: Teresa Lever, Columbia, MO (US); Elsie Barry, Columbia, MO (US); Sam Schaeffer, Columbia, MO (US); Avery Schroeder, Columbia, MO (US)

(21) Appl. No.: 19/054,488

(22) Filed: Feb. 14, 2025

Related U.S. Application Data
(60) Provisional application No. 63/554,887, filed on Feb. 16, 2024.

(51) Int. Cl. A61N 1/36; A61N 1/04

(52) U.S. Cl. CPC A61N 1/36034; A61N 1/0452; A61N 1/0456; A61N 1/36003; A61N 1/3601; A61N 1/36025

(57) ABSTRACT

A TENS-SLN method is disclosed that avoids causing (or minimizes) muscle contractions in the neck and instead sends a neural code to the brainstem to evoke swallowing, which can be readily detected by routinely used clinical methods such as surface laryngeal EMG, transnasal endoscopy, respiratory plethysmography, and/or laryngeal palpation. Moreover, without electrode position change, alteration of the electrical code can evoke the laryngeal adductor reflex (LAR). Thus, TENS-SLN facilitates assessment and/or treatment of two different airway protective reflexes (swallowing and the LAR), particularly when used in combination with existing methods such as surface laryngeal EMG, transnasal endoscopy, respiratory plethysmography, and/or laryngeal palpation. This method entails applying surface electrodes at a specific location (spot on the anterior neck and delivering a precise electrical code to a precise brainstem region via the superior laryngeal nerve (SLN) branch of the vagus nerve to evoke either swallowing or the LAR (i.e., code-specific response).

### CROSS REFERENCE TO RELATED APPLICATIONS

This application claims priority under 35 U.S.C. § 119 (e) to provisional patent application U.S. Ser. No. 63/554,887, filed Feb. 16, 2024. The provisional patent application is hereby incorporated by reference in its entirety herein, including without limitation: the specification, claims, and abstract, as well as any figures, tables, appendices, or drawings thereof.

### TECHNICAL FIELD

The present disclosure relates generally to a noninvasive electrical stimulation method to facilitate the evaluation and treatment of dysphagia. The attached Appendix is herein incorporated by reference in its entirety, including without limitation, any figures, tables, appendices, or drawings thereof.

### BACKGROUND

The background description provided herein gives context for the present disclosure. Work of the presently named inventors, as well as aspects of the description that may not otherwise qualify as prior art at the time of filing, are neither expressly nor impliedly admitted as prior art.

Neurogenic dysphagia (i.e., swallowing impairment caused by neurological disease) affects approximately 500,000 individuals annually in the United States. Common symptoms include malnutrition, dehydration, and respiratory complications (particularly aspiration pneumonia), all of which may result in a poor quality of life and contribute to death in affected individuals. Relatively little is known about the pathogenesis of dysphagia in neurological diseases, and as a result, treatments are primarily symptom-based rather than targeting the underlying problems. Neurogenic dysphagia, therefore, is an important area for research that has the potential to benefit hundreds of thousands of individuals living within the United States (as well as many more living beyond these borders) who are afflicted by various neurological diseases ranging from degenerative, e.g., amyotrophic lateral sclerosis/ALS, Parkinson's, and advanced aging, cerebrovascular, e.g., cerebral ischemia or infarction, congenital, e.g., DiGeorge syndrome and Rett syndrome, and neoplastic processes, e.g., tumors, and their associated surgical and/or medical interventions.

For reasons that remain largely unknown, these diseases/conditions particularly affect the complex neural network that innervates the aerodigestive tract, which includes organs and tissues shared by the respiratory tract and upper digestive tract (i.e., oral cavity, pharynx, and larynx). When these vital upper airway structures fail to work normally, bacteria-laden food/liquid/saliva is allowed to enter the larynx, i.e., “gateway” to the lungs, often resulting in aspiration pneumonia, a life-threatening lung infection associated with considerable economic and societal burden due to high 30-day hospital readmission rates, extended hospital length of stay, accompanying morbidity, and early mortality. While prevention of aspiration pneumonia is a primary healthcare goal, efforts have largely failed in the absence of effective dysphagia treatment strategies to minimize aspiration pneumonia risk.

One known dysphagia treatment is transcutaneous electrical stimulation, which is widely used by medical speech-language pathologists (SLPs) in clinical practice. The current treatment approach is called neuromuscular electrical stimulation (NMES), which entails using surface electrodes on the anterior neck (typically immediately above and/or below the thyroid cartilage) to repetitively contract the muscles involved in swallowing via a hand-held electrical stimulator (FIG. 1). During a typical treatment session, NMES is delivered in continuous trains of 55 seconds ON and 5 seconds OFF for up to 1 hour, using a stimulus frequency of 80 Hertz (Hz) and 700 μs pulse width. During the stimulation ON phase, the muscles beneath the electrodes continually contract, which feels like a continuous strong “grabbing” sensation in the neck until the muscles are allowed to momentarily relax during the brief stimulation OFF phase. Importantly, this NMES approach does not increase the patient's “urge” to swallow. Still, it causes only local muscle contractions rather than evoking a patterned swallowing response (i.e., “peristaltic contractions” to propel a bolus from the mouth to the esophagus for digestion). While the muscles are contracted (i.e., stimulation ON phase), the patient is instructed to repeatedly try their best to dry swallow (i.e., swallow saliva) or swallow sips of liquid from a cup/straw, despite not feeling an increased urge to do so. This treatment approach is typically repeated several days per week over several weeks or months. The underlying premise is that repetitive swallowing against muscle resistance strengthens the “swallowing muscles” and result in improved swallowing function and airway protection. However, there is inconclusive evidence of its effectiveness, which makes NMES for dysphagia a highly controversial topic in clinical practice and research. Moreover, the “standard” stimulus parameters (i.e., high/80 Hz and long/700 μs pulse width) are known to be muscle-fatiguing and thus potentially harmful for patients with neurodegenerative diseases.

The current “peripheral/muscle-focused” dysphagia treatment approach is markedly underdeveloped and thus requires innovation to improve patient outcomes. Thus, there exists a need in the art for dysphagia treatment that improves patient outcomes.

### SUMMARY

Based on the extensive research with rodent models of dysphagia (swallowing impairment), the present disclosure presents a methodology to safely and consistently evoke increased swallow rates and urge to swallow in people, without fatigue or alteration in vital signs. The methodology includes optimized electrical stimulation parameters and evaluation/treatment protocols for use with patients with dysphagia due to a variety of conditions affecting the neural control of swallowing, such as Parkinson's disease, stroke, amyotrophic lateral sclerosis, head, and neck cancer, to name but a few. The method entails applying a pair of commercially available surface electrodes at a specific location, i.e., a spot on the anterior neck, and delivering a precise electrical “code” to the brainstem center pattern generator for swallowing via the superior laryngeal nerve (SLN) branch of the vagus nerve. This combined methodology, which is called TENS-SLN (i.e., transcutaneous electrical nerve stimulation targeting the SLN), produces an immediate treatment effect of repeated swallowing in conjunction with a strong urge to swallow throughout a thirty-to-sixty-minute treatment session. This novel approach is vastly different from the standard electrical stimulation approach currently used by speech-language pathologists for dysphagia therapy, i.e., neuromuscular electrical stimulation or NMES, which entails using surface electrodes to stimulate and strengthen muscles in the neck, without increasing the urge to swallow or actually evoking/triggering swallowing. This TENS-SLN method avoids causing (or minimizes) muscle contractions in the neck and instead sends a “neural code” to the brainstem to evoke the swallowing reflex, which can be readily detected by routinely used clinical methods such as surface laryngeal electromyography (EMG), transnasal endoscopy, respiratory plethysmography, and/or laryngeal palpation. Moreover, without changing the position of the electrodes, the electrical code can be altered to evoke the laryngeal adductor reflex (LAR). Thus, this novel methodology uniquely facilitates the assessment and/or treatment of two different airway protective reflexes (swallowing and the LAR), particularly when used in combination with existing methods such as surface laryngeal EMG, transnasal endoscopy, respiratory plethysmography, and/or laryngeal palpation. While there are numerous commercially available hand-held devices, none can generate the TENS-SLN codes for swallowing and the LAR; therefore, this method relies on programmable research-grade equipment for methodological development and safety/feasibility studies in human subjects. However, this research-grade equipment is too expensive and bulky for routine clinical use, and its complexity renders it non-clinician-friendly. This can be incorporated with the TENS-SLN codes into a simple hand-held device for clinical evaluation and treatment of dysphagia.

The following objects, features, advantages, aspects, and/or embodiments are not exhaustive and do not limit the overall disclosure. No single embodiment need provide each and every object, feature, or advantage. Any of the objects, features, advantages, aspects, and/or embodiments disclosed herein can be integrated with one another, either in full or in part.

It is a primary object, feature, and/or advantage of the present disclosure to improve on or overcome the deficiencies in the art.

It is a further object, feature, and/or advantage of the present disclosure to develop a TENS-SLN protocol to reliably evoke either swallowing or the LAR, as both of these airway protective reflexes can be investigated using a variety of innovative methodologies.

It is still yet a further object, feature, and/or advantage of the present disclosure to pinpoint the SLN, such as in the in the anterior neck for surface stimulation of the SLN (i.e., TENS-SLN).

It is still yet a further object, feature, and/or advantage of the present disclosure to demonstrate that it is possible to reliably and safely stimulate the SLNi using non-invasive surface electrodes, in preparation for future studies with patients with neurogenic dysphagia.

It is still yet a further object, feature, and/or advantage of the present disclosure to reliably localizing the SLNi for precise placement of the surface electrodes for targeted stimulation.

It is still yet a further object, feature, and/or advantage of the present disclosure to achieve rapid and accurate TENS-SLN electrode placement using only digital laryngeal palpation in the majority of cases, which can be critical for clinical adoption of this novel dysphagia treatment.

It is still yet a further object, feature, and/or advantage of the present disclosure to alter the current clinical NMES dysphagia therapy protocol to selectively target the SLNi and upstream brainstem synapses to reliably evoke swallowing rather than merely causing local muscle contractions.

It is still yet a further object, feature, and/or advantage of the present disclosure to determine the TENS-SLN stimulus parameters that can safely and consistently evoke increased swallow rates and urge to swallow in both males and females without fatigue or alteration in vital signs.

It is still yet a further object, feature, and/or advantage of the present disclosure to uniquely facilitate assessment and/or treatment of two different airway protective reflexes (swallowing and the LAR), particularly when used in combination with existing methods such as surface laryngeal EMG, transnasal endoscopy, respiratory plethysmography, and/or laryngeal palpation.

The non-invasive electrical stimulation methods disclosed herein can be used in a wide variety of applications. For example, non-invasive TENS-SLN can be used as a targeted multi-modal treatment for impaired airway protection, such as in patients with dysphagia due to, for example, Parkinson's disease, stroke, amyotrophic lateral sclerosis, head and neck cancer, and other neurological-based diseases/conditions resulting in compromised airway protective reflexes and associated high aspiration pneumonia risk.

It is still yet a further object, feature, and/or advantage of the present disclosure to provide a device that can generate the TENS-SLN codes for swallowing and the LAR disclosed herein. It is preferred that the device be portable, non-invasive and safe to use, cost effective, and durable.

It is still yet a further object, feature, and/or advantage of the present disclosure to continue protocol refinement and optimization for improved workflow efficiency and clinical outcomes for dysphagia patient trials.

For example and with regard to safety, safety of surface stimulation of the anterior neck has been well-established in the literature. Trainings/certifications to provide this dysphagia treatment intervention are beneficial. A doctor's experience in routinely performing electrophysiological studies (cranial nerve stimulation and EMG/EEG and physiologic vital sign recordings) as a major component of translational animal research agenda is also beneficial. Potential risks that have been overcome by the devices disclosed herein include sensitivity of the skin to alcohol-based skin cleanser and skin abrasion gel, mild discomfort during electrical stimulation if the electrodes become detached at any point during testing, and heightened awareness or anxiety during electrical stimulation. Another potential risk overcome by the devices disclosed herein is the alteration in vital signs (e.g., increase/decrease of heart rate/HR or respiratory rate/RR or decrease of blood oxygen saturation levels/SpO2). Regardless, vital signs are monitored during the entire electrical stimulation protocol, and baseline values are obtained before attaching the stimulation electrodes. At any point in time, if a participant experiences a >20% change from baseline HR or RR (in either direction) or SpO2 (decrease) or any of these vital signs falling outside normative published ranges, testing is terminated immediately. In such cases, physiological monitoring is continued for up to 30 minutes to determine if vital signs return to (and stay at) baseline levels and no additional concerns are identified by the research team or reported by the participant. Otherwise, EMS is called for medical evaluation of the participant. In all cases, the participant should be contacted within 24 hours following testing to ensure no further adverse events are reported that would necessitate physician evaluation for medical management. Safety of transnasal endoscopy has been well-established in the literature and a doctor's routine performance of same in their clinical practice and/or research studies to evaluate swallowing and/or the LAR is highly beneficial. Risks and side effects related to the transnasal endoscopy procedure include nosebleed or fainting, which are reported to be rare in the literature. However, in the case of such adverse events, the subject is treated in the standard manner by the endoscopist. Though there are no reported incidences of airway compromise (i.e., blocked airway) associated with transnasal endoscopy, a doctor's training in airway management and all emergency equipment is available during the procedure when performed the clinic can be highly beneficial.

For example and with regard to cost-effectiveness, commercially available equipment and supplies were used as appropriate throughout the protocol described herein. For example, although commercially available electrical stimulation devices were used, the stimulation waveform under investigation in the present disclosure is not the typical one used for the treatment of swallowing problems. Moreover, the methods and/or devices disclosed herein may replacement and/or supplement procedures that are not billable to the participants' medical insurance because they are deemed “healthy” individuals. Therefore, the standard of care can become to have no procedures performed.

Further methods can be practiced which facilitate use, manufacture, assembly, maintenance, and repair of said devices which accomplish some or all of the previously stated objectives. Said devices can further be incorporated into systems or kits which accomplish some or all of the previously stated objectives. In one such example, the aforementioned devices can be used on patients in conjunction with having the patients take a nasal decongestant that temporarily relieves congestion and swelling in the nose to open nasal passages for transnasal laryngoscopy.

According to some aspects of the present disclosure, a method of evoking swallowing or the laryngeal adductor reflex comprises applying a pair of surface electrodes at a specific location on an anterior neck; and delivering an electrical code a brainstem region via the superior laryngeal nerve (SLN) branch of the vagus nerve to evoke either swallowing or the laryngeal adductor reflex (LAR).

According to some additional aspects of the present disclosure, the LAR is a code-dependent response.

According to some additional aspects of the present disclosure, the method further comprises targeting an internal branch of the SLN (SLNi) and upstream brainstem synapses to reliably evoke swallowing rather than merely causing local muscle contractions.

According to some additional aspects of the present disclosure, the method further comprises moving the stimulating electrodes laterally over the larynx and to selectively stimulate the SLNi.

According to some additional aspects of the present disclosure, the method further comprises positioning the stimulating electrodes parallel with the SLNi to drive an electrical current flow toward the brainstem and away from the muscles in the neck.

According to some additional aspects of the present disclosure, the method is free from using surface electrodes to stimulate and strengthen muscles in the neck without evoking an urge to swallow.

According to some other aspects of the present disclosure, a therapeutic method comprises the method according to any one or more of the preceding paragraphs and utilizing TENS-SLN to selectively treat multiple airway protective reflexes.

According to some other aspects of the present disclosure, a diagnostic method further comprises diagnosing a health condition based on detection of a neural response to the delivery of the precise electrical code to the corresponding region of the brainstem.

According to some additional aspects of the present disclosure, the diagnostic method further comprises accomplishing said detection utilizing surface laryngeal electromyography.

According to some additional aspects of the present disclosure, the diagnostic method further comprises accomplishing said detection utilizing transnasal endoscopy.

According to some additional aspects of the present disclosure, the diagnostic method further comprises accomplishing said detection utilizing respiratory plethysmography.

According to some additional aspects of the present disclosure, the diagnostic method further comprises accomplishing said detection utilizing laryngeal palpation.

According to some additional aspects of the present disclosure, the diagnosis comprises dysphagia due to a condition selected from the group consisting of: (i) Parkinson's disease; (ii) stroke; (iii) amyotrophic lateral sclerosis; and (iv) cancer.

According to some other aspects of the present disclosure, a handheld transcutaneous electrical nerve stimulation (TENS)/neuromuscular electrical stimulation (NMES) unit that evokes swallowing or the laryngeal adductor reflex comprises a portable body; a display; an electrical stimulator including: a pair of surface electrodes that can be attached to a mammalian body; and a transmitter that allows for pulsing an electrical code to a location of a brainstem region of the mammalian body. The location depends on information within the electrical code, thereby allowing an operator of the handheld TENS/NMES unit control over which location of the brainstem region that the electrical code is sent to.

According to some additional aspects of the present disclosure, the handheld TENS/NMES unit comprises a transcutaneous electrical nerve stimulation superior laryngeal nerve (TENS-SLN) unit.

According to some additional aspects of the present disclosure, the handheld TENS/NMES further comprises a user interface that allows the operator to input a stimulus frequency between 0.1 and 100 Hz.

According to some additional aspects of the present disclosure, the handheld TENS/NMES further comprises a user interface that allows the operator to input a pulse width between one hundred microseconds (100 μs) and one millisecond (1 ms).

According to some additional aspects of the present disclosure, the handheld TENS/NMES further comprises a user interface that allows the operator to input a stimulus polarity selected from the group consisting of: (i) anodal and/or toward the brain; and (ii) cathodal and/or toward the muscles.

According to some additional aspects of the present disclosure, the handheld TENS/NMES further comprises a user interface that allows the operator to input a length of a duration and/or a pattern of the pulsing.

According to some additional aspects of the present disclosure, the display is configured to output participant data selected from the group consisting of: (a) sensory threshold intensity (mA); (b) therapeutic stimulus intensity (mA); (c) change in swallow frequency (swallows per minute); (d) swallow latency; (e) respiratory rate; (f) heart rate; and (g) blood oxygen saturation, from baseline values.

A principal benefit to the community includes a potentially safe and effective non-invasive treatment to improve airway protective reflexes (e.g., swallowing and the laryngeal adductor reflex).

Some other benefits and/or advantages of the present disclosure include that there may be a direct medical benefit to the participant, the participant may benefit from a free laryngeal examination, and the participant's participation may contribute to the supervising doctor's medical knowledge.

The information presented herein can lead to the development of TENS-SLN into a safe and effective non-invasive treatment for patients with neurogenic dysphagia.

These and/or other objects, features, advantages, aspects, and/or embodiments will become apparent to those skilled in the art after reviewing the following brief and detailed descriptions of the drawings. The present disclosure encompasses (a) combinations of disclosed aspects and/or embodiments and/or (b) reasonable modifications not shown or described.

### BRIEF DESCRIPTION OF THE DRAWINGS

Several embodiments in which the present disclosure can be practiced are illustrated and described in detail, wherein like reference characters represent like components throughout the several views. The drawings are presented for exemplary purposes and may not be to scale unless otherwise indicated.

FIG. 1 shows an example of anterior neck electrode placement currently used for NMES dysphagia treatment. Electrode pairs are positioned on the anterior neck for surface electrical stimulation (80 Hz, 700 μs pulse duration). The goal is to contract the “swallowing muscles” to strengthen them, rather than evoking the swallowing reflex.

FIG. 2 illustrates a neural swallowing circuit. Swallowing is a complex sensori-motor reflex that involves 6 cranial nerves (CNs), a brainstem central pattern generator (CPG), and 5 brainstem motor nuclei.

FIGS. 3A-3E show a therapeutic effect of SLNi stimulation in a transgenic mouse model of ALS. As shown in FIG. 3A, an invasive survival surgery procedure was performed in a mouse model of ALS (SOD1-G93A) at 6 months of age (i.e., clinical disease onset) to apply direct electrical nerve stimulation to the SLNi (DENS-SLN) in the right side of the neck. FIG. 3B shows a miniature bipolar electrode for DENS-SLN beside a US penny for size comparison. Note the Teflon shield cradling the electrodes to prevent current spread. FIG. 3C shows an intra-operative view of the SLNi on the electrodes, which are oriented for anodal stimulation (i.e., to direct the current centrally toward the brainstem instead of peripherally to the muscles). FIG. 3D shows DENS-SLN consistently evoked an average of 2 swallows per 20 second stimulus train, without “swallow decay” over a 30-minute treatment session. As shown in FIG. 3E, this single “therapeutic session” nearly preserved licking (and swallowing) function at disease end-stage (˜9 months of age). Asterisk: p<0.05; error bars=SEM.

FIG. 4 shows a non-invasive TENS-SLN approach to evoke swallowing. The left side of the figure shows key laryngeal structures are drawn on the anterior neck of an adult male for anatomical reference. The right side of the figure shows a stereotypical location of the SLN is shown in the anterior neck, with its internal (sensory, SLNi) and external (motor, SLNe) branches flanking above and below the thyroid cartilage, respectively. Note the surface electrodes for TENS-SLN is positioned directly over (and in parallel) with the SLNi and oriented for anodal stimulation to “drive” the electrical current flow toward the brainstem and away from the muscles in the neck.

FIG. 5 shows TENS-SLN pattern evokes swallowing. Each block of electrical stimulation is divided into 20-30-second trains, with each train consisting of 10 seconds of rest (NO stimulation) followed by 15-20 seconds of 40 Hz stimulation lasting up to 5 minutes. R=rest; S=stimulation.

FIGS. 6A-6B shows a non-invasive laryngeal EMG. FIG. 6A shows a schematic shows the location of surface EMG electrodes on the anterior neck skin, with subcutaneous anatomic structures shown for reference. Note there is 2-4 electrodes paired in various configurations to capture thyroarytenoid (left/right thyroid cartilage) and cricothyroid (left/right cricoid cartilage) EMG activity versus left/right laryngeal EMG activity (left thyroid+left cricoid and right thyroid+right cricoid) versus global laryngeal EMG activity (e.g., horizontal or crisscross configuration), as shown by the yellow double arrows between the electrodes. FIG. 6B shows the yellow dashed box shows the corresponding EMG electrode placement on the anterior neck skin of an adult male.

FIGS. 7A-7B show laryngeal brainstem evoked potentials (LBEPs) in laboratory rodents. FIG. 7A shows a schematic of optimized EEG electrode montage for recording LBEPs in lightly anesthetized rodents via subcutaneous needle electrodes. FIG. 7B shows evoked swallowing activity is extracted from the background EEG recording to generate an averaged LBEP waveform consisting of a series of 8 peaks that are time-locked to EMG swallow bursts. Early peaks correspond with sensory components of the neural swallowing circuit (i.e., Peak 1=SLNi; Peaks II & III=brainstem central pattern generator interneuron pools), whereas later peaks (IV-VIII) correspond with brainstem motor neuron pools generating EMG swallow activity.

FIG. 8 shows an EEG electrode montage for recording brainwaves. To record brainwave activity (for stress level and LBEP assessment), EEG electrode pairs are attached to the skin on the high forehead and over the mastoid process on each side of the head. A ground electrode are attached to the skin over the 7th cervical vertebra.

FIG. 9A-9D show transnasal endoscopy. FIG. 9A shows the endoscopic is advanced just above the epiglottis to permit visualization of the bilateral larynx during airway reflex testing. The yellow asterisk denotes the glottal space between the bilateral vocal folds (VFs), which are open (abducted) during breathing. FIG. 9B shows during the LAR, the glottis abruptly closes (i.e., the VFs adduct/approximate at midline, denoted by black asterisk) for ˜½ second before reopening to resume breathing. FIGS. 9C-9D show during swallowing, the glottic closes (FIG. 9C; black asterisk), and the larynx becomes obscured (FIG. 9D) as the epiglottis inverts to protect the airway as the entire larynx elevates (i.e., moves closer to the endoscope camera).

FIG. 10 shows a detailed flowchart of an example TENS-SLN protocol. After obtaining baseline swallowing rate and vital signs, TENS-SLN optimization ensued.

FIGS. 11A-11C show optimized electrode placement for TENS-SLN treatment. FIG. 11A evidences better anatomical landmark identification, FIG. 11B evidences improved accuracy in placement of electrodes in the carotid triangle where the SLN is situated, and FIG. 11C evidences an increase in the interphase delay from 50 μs to 100 μs are factors that contributed to effective optimization of the TENS-SLN protocol.

FIG. 12 shows that multiple stimulus intensities (between 5-10 mA) were tested to determine the therapeutic level that elicited 2-4 swallows per 20 second stimulus train, identified by laryngeal EMG peaks (red stars) in synchrony with laryngeal elevation (via webcam recording) and disrupted respiratory pattern (i.e., brief apnea).

FIGS. 13A-13C show there were no significant changes in vital signs during TENS-SLN trials. Respiratory rate (FIG. 13A), heart rate (FIG. 13B), and SpO₂ (FIG. 13C) remained within normal limits for each participant throughout each session.

FIG. 14A shows all 9 participants demonstrated a similar response pattern during TENS-SLN—an initial abrupt increase in swallow rate that was sustained across treatment trials, followed by a return near baseline values. FIG. 14B shows a participant report of “Urge to Swallow” had a similar profile. Asterisks denote statistical significance (p<0.05; RMANOVA with Bonferroni posthoc comparisons). Dashed line=mean; gray shading=SEM. T1=Trial 1; T2=Trial 2; and T3=Trial 3. For effortful swallows, baseline and endline=no SLN stimulation.

FIGS. 15A-15B shows the effect of SLN stimulation on swallowing in ALS mice. FIG. 15A shows SLN stimulation in 6-month ALS mice via an invasive surgical procedure consistently evoked 2-4 swallows per 20-second stimulus train. FIG. 15B shows most swallows occurred within the first 8 seconds after initiating each 20-second stimulus train.

FIG. 16 shows electrophysiological recording during SLN stimulation.

FIG. 17A shows TENS-SLN significantly increased saliva swallow rate compared to baseline (p<0.0001, ***). FIG. 17B shows swallow rates were compared between each 20-second stimulation train throughout a single 5-minute treatment session, which revealed no significant differences (p=0.58; ANOVA); black line=mean; gray shading=SEM.

FIG. 18 shows swallow latencies were compared between each 20-second stimulation train throughout a single 5-minute treatment session, which revealed no significant differences (p=0.98; ANOVA); black line=mean; gray shading=SEM.

FIGS. 19A-19B compare respiratory rate (FIG. 19A) and heart rate (FIG. 19B) between each 20-second stimulation train throughout a single 5-minute treatment session, which revealed no significant differences (p=0.88 and p=0.76, respectively; ANOVA). FIG. 19C shows there was no significant difference (p=0.58; ANOVA) in SpO₂ from the Block Start to Block End. Black line=mean; gray shading=SEM.

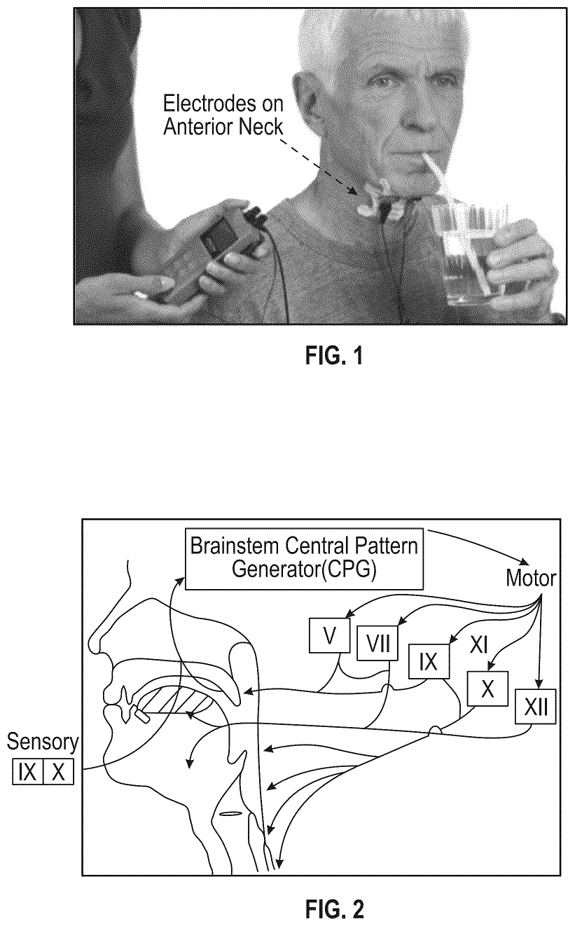

An artisan of ordinary skill in the art need not view, within isolated figure(s), the near infinite distinct combinations of features described in the following detailed description to facilitate an understanding of the present disclosure.

### DETAILED DESCRIPTION

The present disclosure is not to be limited to that described herein. Mechanical, electrical, chemical, procedural, and/or other changes can be made without departing from the spirit and scope of the present disclosure. No features shown or described are essential to permit basic operation of the present disclosure unless otherwise indicated.

As shown in FIG. 2 and Table 1, swallowing is a complex sensori-motor reflex that relies on several “upstream” sensory (afferent) and central pattern generator (CPG) components that are largely ignored by the current NMES clinical approach. There is a growing body of research (including the own laboratory animal research) showing that swallowing cannot occur without stimulation of the internal branch of the superior laryngeal nerve (SLNi) of the vagus (cranial nerve 10; CNX, see FIG. 2 and Table 1 below), which contains sensory (i.e., afferent) fibers innervating the base of the tongue, epiglottis, and mucous membranes of the larynx as far inferiorly as the vocal folds (VFs). Based on extensive experimental research over the past decade with laboratory mice, SLNi stimulation parameters have been identified that reliably evoke swallowing without causing adverse events.

TABLE 1
Neural Control of the Swallowing Reflex

| CN Afferents - | | | | |
|---|---|---|---|---|
| SENSORY | Brainstem Interneurons | | CN Efferents - MOTOR | |
| Petrosal (CN | Swallow | Nucleus tractus solitarius | Brainstem | Trigeminal (pons; via CN |
| IX) Carries | Central | (NTS) (Dorsal | Motor | V) Innervates submental |
| sensory | Pattern | Swallowing Group) | Nuclei | muscles |
| information | Generator | | | Facial (pons; CN VII) |
| from the soft | | | | Innervates facial muscles |
| plate, posterior | | Para-nucleus ambiguous | | Nucleus ambiguous |
| 1/3 tongue, and | | (Para-NA) (Ventral | | (medulla; via CN IX, X, |
| pharynx | | Swallowing Group) | | XII) |
| Nodose (CN X) | | | | Dorsal motor nucleus of |
| Carries sensory | | | | the vagus (medulla; via |
| information | | | | CN X) Innervates |
| from the | | | | laryngeal muscles |
| pharynx and | | Reticular Formation | | Hypoglossal (medulla; via |
| larynx via | | | | CN XII) Innervates to |
| laryngeal nerves | | | | tongue molecules |

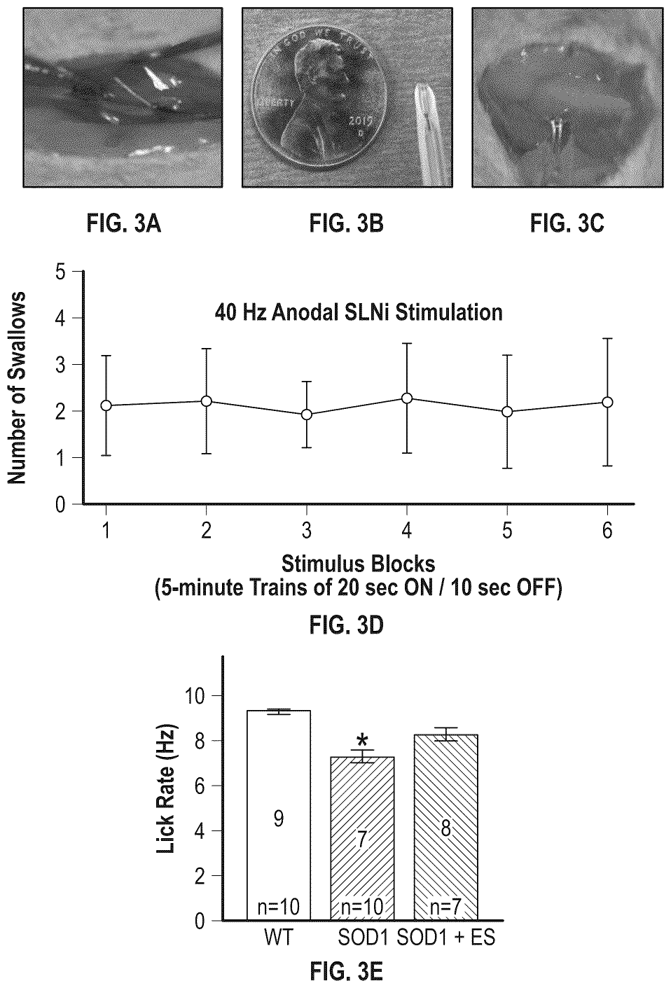

Specifically, a survival surgery approach directly stimulated the SLNi in a mouse model of ALS (FIGS. 3A-C) while investigating several stimulus parameters, including stimulus rate (Hertz or Hz), pulse width (μs), stimulus polarity (anodal/toward the brain versus cathodal/toward the muscles), stimulus train ON/OFF duration pattern (sec). The results revealed that 40 Hz anodal (i.e., directed toward the brainstem), charge balanced rectangular pulses (200-400 μs) delivered in continuous trains of 20-seconds ON, 10-seconds OFF consistently evoked an average of 2 swallows per 20 second stimulus train (i.e., ˜8 swallows per minute) without evidence of “swallow decay” (FIG. 3D) or adverse effects on respiratory rate or heart rate (data not shown). Moreover, a single treatment at 6 months of age remarkably maintained licking/swallowing function in ALS mice as the disease advanced (assessed via videofluoroscopic swallow study), compared to untreated controls (FIG. 3E). Collectively, the preclinical animal research demonstrates an untapped therapeutic benefit of targeted SLNi stimulation for dysphagia, which provides robust rationale to proceed with clinical translation to humans.

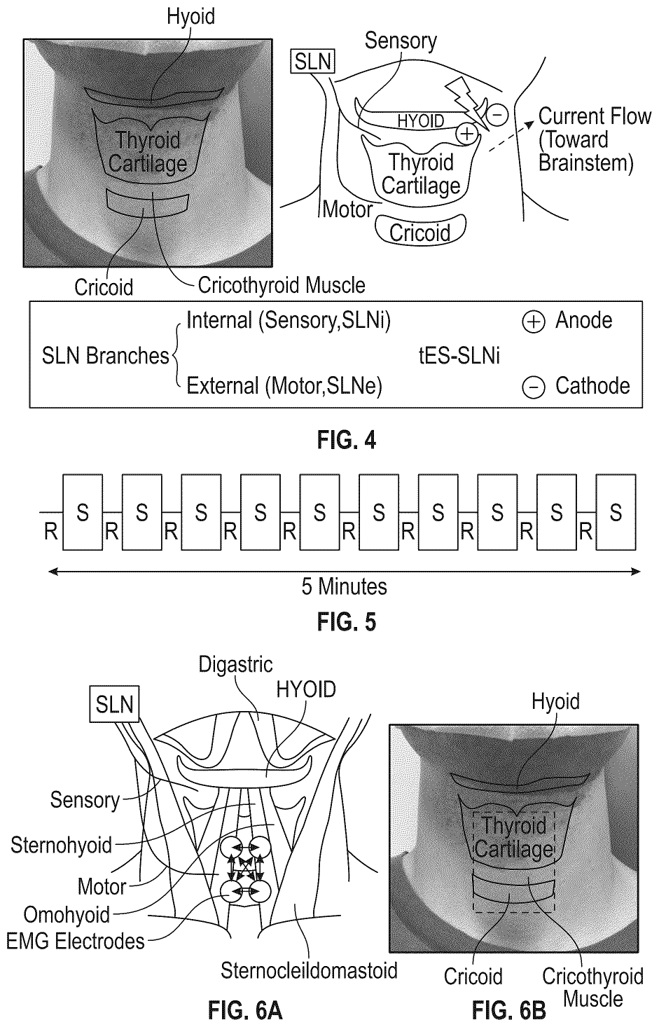

The current clinical NMES dysphagia therapy protocol can be altered to selectively target the SLNi and upstream brainstem synapses to reliably evoke swallowing rather than merely causing local muscle contractions. The targeted approach is called TENS-SLN (i.e., transcutaneous electrical nerve stimulation targeting the SLNi), which simply entails moving the stimulating electrodes more laterally over the larynx to selectively stimulate the SLNi (FIG. 4) while delivering the experimental 40 Hz (i.e., less fatiguing) anodal stimulation parameters.

Numerous human laryngeal cadaveric dissections in the gross anatomy lab at the MU School of Medicine were conducted to repeatedly identify the SLNi in both sides of the neck, which run in a stereotypical fashion between the hyoid bone and thyroid cartilage to pierce through the thyrohyoid membrane and thyrohyoid muscle to provide sensory innervation to the pharynx and larynx. The exact course the SLNi takes between these two laryngeal anatomical landmarks is quite variable (up to 2 cm variability). Therefore, a combination of digital laryngeal palpation (i.e., finger/thumb compression of the laryngeal structures) and ultrasound guidance can facilitate accurate and fast electrode placement over the SLNi. Indeed, ultrasound identification of the SLNi in human subjects has been successfully (i.e., 100% of subjects) and quickly (<2 minutes per subject) performed by several research groups (for example) for targeted SLNi nerve blocking procedures to aid awake endotracheal intubation in patients with a difficult airway. Moreover, ultrasound facilitates identification of laryngeal anatomical landmarks in subjects with excessive fatty neck tissue that hinders digital laryngeal palpation.

Rapid and accurate TENS-SLN electrode placement uses only digital laryngeal palpation in the majority of cases, which can be critical to clinical adoption of this novel dysphagia treatment. This feature can further aid in reliably localizing the SLNi for precise placement of the surface electrodes for targeted stimulation.

In addition to swallowing, it is known from a growing body of experimental animal research (see e.g., Lever, et al., “A mouse model of pharyngeal dysphagia in amyotrophic lateral sclerosis.” Dysphagia, 2010. 25(2): p. 112-26, and U.S. Pat. No. 10,542,911, which are hereby incorporated by reference in their entireties herein) and a few clinical research studies that SLNi stimulation can evoke a variety of upper airway protective reflexes. That is, electrical stimulation of the SLNi can be “tuned” to evoke either swallowing, the laryngeal adductor reflex (LAR), coughing, sneezing, etc., to protect the airway from foreign material, depending on the stimulus frequency used. Specifically, stimulation between 10 and 50 Hz typically evokes reflexive swallowing in healthy animals, whereas stimulation<10 Hz may produce sneezing and coughing, and stimulation<1 Hz evokes the LAR (i.e., abrupt closure of the vocal folds for ˜½ second). TENS-SLN can thus selectively treat multiple airway protective reflexes for improved dysphagia management and healthcare outcomes.

### Examples

The present disclosure develops a TENS-SLN protocol to reliably evoke either swallowing or the LAR.

Compared to the complex sensori-motor neural swallowing circuit that involves 6 cranial nerves, the LAR is controlled by a relatively simple sensori-motor neural circuit involving only the vagus nerve. Both of these airway protective reflexes are commonly pathological in neurological diseases, thus targeted TENS-SLN may provide an effective multi-modal treatment approach for dysphagia. To evoke the LAR, 0.2-0.5 Hz stimulation (i.e., 1 stimulus every 2-5 seconds) was delivered, which was sufficiently spaced to prevent alteration in respiratory rate.

In addition, the stimulus pulse width can be increased to 1 ms to match published studies using percutaneous needle stimulation of the SLNi to evoke the LAR. The results represent the first exploration of non-invasive TENS-SLN, but are also the first to simultaneously investigate TENS-SLN-evoked swallowing and the LAR. The improvements of the present disclosure is thus significant in that they can serve the foundation to develop TENS-SLN as a targeted multi-modal treatment for impaired airway protection, first in patients with Parkinson's disease, and ultimately other neurological-based diseases/conditions resulting in compromised airway protective reflexes and associated high aspiration pneumonia risk.

A brief overview of an example study is provided here, followed by a more detailed description below. In brief, each subject participated in up to 4 test sessions (on separate days) lasting up to 2 hours each, as summarized in Table 2. All 4 sessions were spaced at least 2 days apart (i.e., emulating the typical clinical visit schedule) and completed within a 3-month timeframe to accommodate subject/research personnel availability. These multiple sessions are necessary to optimize the TENS-SLN electrode placement and stimulus settings for each subject as well as to demonstrate repeatability in a subset of subjects (25-50% of subjects). Following study consent, the participant completed two questionnaires (health history/status and EAT-10) to verify eligibility to participate in the study before proceeding with the test protocol. Prior to initiating TENS-SLN trials, baseline electrophysiological recordings of vital signs, laryngeal electromyographic (EMG) activity, and (in a subset of subjects) electroencephalography (EEG) activity were acquired via surface electrodes/sensors connected to electrophysiological recording equipment. Following baseline electrophysiological recordings, the TENS-SLN procedure began. First, a combination of digital laryngeal palpation (and/or ultrasound as needed) was used to facilitate accurate placement of a pair of stimulating electrodes on the anterior neck for targeted SLNi stimulation. Once accurate electrode placement is confirmed, TENS-SLN was delivered in up to 5-minute blocks (in synchrony with electrophysiological recordings) to allow frequent “rest breaks” for safety/tolerance re-assessment, with “hard stop” criteria outlined below. Only single-use skin preparation supplies and electrode/transducer gels was used to prevent cross-contamination between participants. Importantly, all essential equipment/supplies are possessed and a bench testing/troubleshooting for protocol workflow has been completed. Therefore, successful completion of this protocol is summarized in the RESULTS section below. See infra.

TABLE 2
Study Design

| | |
|---|---|
| Aim | Demonstrate the feasibility and safety of TENS-SLN-evoked swallowing |
| | versus LAR events. |
| Subjects | 60 healthy adults; ~50:50 males/females; 3 age groups: 20-39, 40-59, 60+ |
| | years old. |
| Primary | Feasibility: |
| Outcomes | a) Can the SLNi be consistently localized in the anterior neck for targeted |
| | TENS-SLN that reliably evokes either swallowing or the LAR, |
| | depending upon the stimulus parameters used? |
| | b) Can TENS-SLN be successfully performed without ultrasound |
| | guidance? |
| | c) Can TENS-SLN be successfully repeated in the same subject on |
| | different days? |
| | Safety: |
| | Can TENS-SLN be tolerated without adversely affecting physiological |
| | vital signs (i.e., respiratory rate, heart rate, and blood oxygen level)? |
| Methods | Feasibility: |
| | a) Questionnaires (i.e., health and swallowing function status) |
| | b) Localization of the SLNi via digital laryngeal palpation (and |
| | ultrasound if needed) |
| | c) Non-invasive TENS-SLN to evoke swallowing or the LAR |
| | d) Non-invasive electrophysiology recordings (vital signs & laryngeal |
| | EMG for all subjects, and EEG for a subset of subjects) |
| | e) Transnasal endoscopy in a subset of subjects for confirmation of |
| | swallowing versus LAR events in the laryngeal EMG recordings |
| | Safety: |
| | Can TENS-SLN be tolerated without adversely affecting physiological |
| | vital signs (i.e., respiratory rate, heart rate, and blood oxygen level)? |

### Questionnaires

After obtaining consent for study participation, each participant completed two self-administered questionnaires pertaining to health and swallow function status to confirm they are healthy participants before proceeding with the study protocol. The first is a health history/status questionnaire developed by the present inventors, whereas the second is the EAT-10 questionnaire, which has been validated as an effective dysphagia screening tool. Both of these questionnaires are included in attached appendix. Subjects who report current or past medical history of exclusionary criteria (e.g., neurological disease, heart disease, head/neck cancer, GERD) was excluded from further testing, as were subjects who score>3 on the EAT-10 questionnaire (i.e., indicative of dysphagia). Excluded participants were replaced by newly recruited subjects until the target study sample size of 60 is reached.

### Localization of the SLNi Via Digital Laryngeal Palpation and Ultrasound

The location of the SLNi were initially estimated based on its stereotypical anatomical trajectory between the hyoid bone and thyroid cartilage, as shown previously in FIG. 4. Following cleaning of the anterior neck with an alcohol pad, the borders of the hyoid bone and thyroid cartilage was identified bilaterally in the anterior neck via digital laryngeal palpation (with gloved hands), followed by drawing the borders on the skin using a surgical skin marker. A commercially available ultrasound device was used to verify these laryngeal landmarks as well as to visualize the location of the SLNi between these landmarks on both sides of the neck, closely following published methods established with human subjects.

Throughout the digital palpation and ultrasound procedures, the participant was seated upright or partially reclined with the neck in a neutral or slightly extended position. For ultrasound-guided SLNi localization, ultrasound gel (single-use, water-based, hypoallergenic, room temperature) was applied to the anterior neck. Using aseptic precautions, the probe was placed longitudinally over the submandibular area to visualize the hyperechoic hyoid bone and thyroid cartilage within the midline. The probe was moved laterally revealing the greater cornu of the hyoid bone and superior horn of the thyroid cartilage. The superior laryngeal artery and its associated SLNi track just medially to these two landmarks. The hyoid and larynx were also palpated and traced on the anterior neck skin for anatomical reference during electrode application. Following ultrasonic visualization and corresponding digital image capture, the gel was removed from the neck with a gauze pad. The distance between the SLNi and the greater cornu of the hyoid bone and superior horn of the thyroid cartilage was measured in the ultrasound images (using the proprietary ultrasound software and/or NIH ImageJ software). The corresponding location and course of the SLNi between these landmarks was precisely measured/identified using digital calipers and traced on the skin using a surgical skin marker, thus allowing for targeted placement of the electrodes directly over the SLNi for TENS-SLN.

### Non-Invasive TENS-SLN to Evoke Swallowing or the LAR:

Following re-cleaning of the anterior neck skin with an alcohol pad, a pair of surface electrodes were applied over the drawn location of the SLNi based on digital laryngeal palpation and ultrasound results. Importantly, the SLNi is the only nerve that traverses between the hyoid and thyroid cartilage where the stimulating electrodes was placed; thus, off-target stimulation of other cranial or cervical spinal nerves is highly unlikely when delivering TENS-SLN. Moreover, anodal stimulation (i.e., positive/anode electrode positioned medial to the negative/cathode electrode to “drive” the electrical current toward the brainstem and away from the muscles) was delivered to activate the upstream neural networks involved in swallowing and the LAR; thus, there should be no “off target” muscle contractions visible in the anterior neck if accurately targeting the SLNi for stimulation. For all participants, a commercial electrical stimulation device was used that permits programing of the target TENS-SLN parameters. For swallowing, the target TENS-SLN parameters comprised 40 Hz stimulation (charge balanced biphasic pulses, up to 400 μs pulse duration), identical to the experimental mouse SLNi stimulation parameters. For the LAR, the target TENS-SLN parameters comprised 0.2-0.5 Hz stimulation (i.e., 1 pulse every 2-5 seconds) using a larger pulse width, in accordance with percutaneous SLNi studies to evoke the LAR in humans.

Before the TENS-SLN “treatment protocol” began, stimulus intensity incrementally increased in 0.1-1 milliamp (mA) steps to first identify the sensory threshold (i.e., the lowest stimulus intensity perceived by the participant), in accordance with the standard clinical protocol for “swallowing muscle” stimulation. This step should take less than 5 minutes. From this point, stimulus intensity continued to be incrementally increased in 0.1-1 mA steps until a therapeutic level is reached. For swallowing, the therapeutic level was indicated by an abrupt increase in swallow rate that does not increase any further with additional increases in stimulus intensity (up to five 1-mA steps), in accordance with the rodent model experiments. For the LAR, the therapeutic level was indicated by a 1:1 stimulus-response rate that does evoke other SLN-mediated reflexes (i.e., swallowing, coughing, etc.) or visible contractions of the anterior neck muscles during each stimulus. Up to 10 trials on either side of the neck may be performed per participant to identify the TENS-SLN “sweet spot” for evoking swallowing and the LAR before proceeding with the “treatment protocol” (described below). Following each TENS-SLN “sweet spot” trial, the patient's perceived pain/discomfort was assessed using the Wong-Baker FACES Scale, where 0=no pain/discomfort and 10=worst pain/discomfort imaginable. For both of these airway protective reflexes, the response rate was determined via laryngeal EMG event detection (described below). In all cases, stimulus intensity did not exceed 25 mA, which is the maximum intensity level routinely used for anterior neck stimulation to treat dysphagia. In cases of no or limited swallowing or LAR events, visibly obvious or patient-perceivable neck/jaw off-target muscle contractions during TENS-SLN, or a pain/discomfort score of ≥4, the stimulation electrodes were repositioned, followed by re-testing of threshold versus therapeutic stimulus levels. Failure to identify the TENS-SLN “sweet spot” within this 10-trial limit resulted in cessation of the test procedure for that particular participant. This “TENS-SLN optimization” process to identify the “sweet spot” may last up to 20 minutes per subject.

Following successful identification of the TENS-SLN “sweet spot”, each participant participated in a single “therapeutic session” consisting of TENS-SLN-evoked swallowing versus LAR, each for up to 15 minutes per side (left/right SLNi). Thus, each participant may receive up to 60 minutes of TENS-SLN in a single session to mimic the typical duration for dysphagia therapy in the clinical setting. A brief (1-2 minute) break between blocks provided sufficient time for re-assessment of changes in vital signs, pain/discomfort rating, and electrode contact between blocks for “go/no go” decision making. During the inter-block break, each subject swallowed a 1-3 mL bolus of bottled water (room temperature, via syringe or self-administered via a medicine cup) to maintain oral hydration and promote normal salivary flow throughout the test procedure. The trial order (swallowing/40 Hz stimulation versus LAR/0.2-0.5 Hz stimulation) and side (left/right) was counterbalanced between successive participants to enhance the study's internal validity. For swallowing, each stimulus block (up to 5 minutes) comprised continuous 20-30-second trains of 15-20 seconds ON, 10 seconds OFF, personalized as needed per subject for success (FIG. 5).

For the LAR, each stimulus block (up to 5 minutes) comprised continuous 0.2-0.5 Hz stimulation (i.e., one stimulus every 2-5 seconds). These stimulus rates should be sufficient to evoke repetitive swallowing versus the LAR, without interfering with breathing (i.e., no significant change in respiratory rate). Throughout the “treatment session”, the participant was comfortably seated in an exam chair facing a computer monitor for visual distraction (e.g., playing a “fish tank” video or “nature documentary video”, either with or without headphones. Once TENS-SLN begins, its effectiveness was evaluated by calculating the change in swallowing versus LAR event rates between baseline and each successive stimulus block for each side (left/right SLNi). The subject's tolerance was monitored throughout the procedure via the pain/discomfort scale, with a score of ≥4 necessitating reassessment of electrodes (i.e., for good skin contact) before proceeding, and a score of 26 necessitating cessation of the test protocol due to intolerance. Once unilateral TENS-SLN is successfully (and safely, see below) demonstrated in several participants, bilateral synchronous TENS-SLN was administered in up to 10 participants for comparison with unilateral stimulation. In some (up to 20) cases, transnasal endoscopy (described below) was synchronously performed during the entire test procedure to conclusively identify and distinguish between swallowing and LAR events in the laryngeal EMG recordings.

According to one example of using the pulse electrical stimulation waveform of FIG. 5, an anodal 40 Hz stimulation using a 225 μs pulse duration (charge balanced, 100 μs pulse width, 25 μs interphase delay) was delivered at an average stimulus intensity of 8 mA (i.e., ˜2× the sensory threshold level reported by the participant) in 5-minute trials divided into 30-second trains, each consisting of 10 seconds of rest (NO stimulation) followed by 20 seconds of 40 Hz stimulation.

### Non-Invasive Electrophysiology

Physiologic vital signs, laryngeal EMG, and EEG activity was recorded in synchrony with TENS-SLN for each participant. Each of these non-invasive electrophysiology-based methods is described in detail below. All electrophysiologic data was displayed in real-time on a computer monitor as well as digitally recorded for more detailed post hoc analysis.

### Physiologic Vital Sign Monitoring

Safety of the TENS-SLN procedure was continually evaluated via physiological monitoring of vital signs. A respiratory sensor (taped to chest or abdomen), electrocardiogram (ECG) sensor (attached to thumb or finger), and pulse oximeter sensor (attached to thumb or finger) was used to measure respiratory rate (RR), heart rate (HR), and blood oxygen saturation (SpO2), respectively. Before attaching the TENS-SLN electrodes, a 5-minute baseline recording was performed to verify each participant is within normal resting ranges for RR (12-20 breaths per minute), HR (60-100 beats per minute), and SpO2 (95-100%), which is expected because healthy adult participants were recruited. Baseline recording began after the participant becomes engaged in watching a nature documentary of their choice as a mental distraction to promote relaxation. Participants falling outside normative resting ranges for one or more physiologic parameters was excluded from further testing and instructed to contact their primary care physician or visit an urgent care center for medical evaluation. Participants with normative ranges proceeded with the test protocol. During each successive TENS-SLN trial, vital sign values (i.e., average per minute) was compared with baseline average values to calculate the percent change from baseline. Based on the extensive animal research as well as published human studies using invasive percutaneous needle stimulation of the SLNi, no adverse effects of non-invasive TENS-SLN on vital signs occurred. However, a >20% change was considered from baseline HR or RR (in either direction) or SpO2 (decrease) or any of these vital signs falling outside normative published ranges as an IRB-reportable adverse event requiring immediate cessation of testing. In such cases, physiological monitoring was continued for up to 30 minutes to determine if vital signs return to (and stay at) baseline levels and no additional concerns are identified by the research team or reported by the participant.

### Laryngeal Electromyography (EMG)

Laryngeal EMG is essential to quantifying swallow and LAR event rates for feasibility outcomes. The EMG procedure entailed attaching 1-2 pairs of electrodes on the anterior neck over the thyroid and/or cricoid cartilages (FIGS. 6A-6B) for recording thyroarytenoid (i.e., vocal fold) and cricothyroid muscle activity, respectively, following standard skin preparation for electrode placement (i.e., cleaning with an ethanol pad followed by skin abrasion gel). The anterior neck was synchronously video recorded (i.e., de-identified; no facial recording) for visual confirmation of laryngeal elevation during EMG swallowing events as well as detection of off-target muscle contractions during stimulation trials to evoke swallowing or the LAR. From the EMG recordings, swallowing versus LAR rates were calculated at baseline versus successive 5-minute TENS-SLN blocks. TENS-SLN resulted in a significant increase in basal swallow rate (e.g., spontaneous swallows to manage saliva; #swallows/minute) and a consistent LAR response (e.g., 1:1 stimulus-response rate), and results were similar between sides but enhanced by bilateral synchronous stimulation due to increased “drive” to the neural networks controlling swallowing and the LAR. Swallowing and LAR response rates did not “decay” throughout the entire test duration, indicative of a sufficiently long treatment session window for future clinical trials.

### Electroencephalography (EEG)

The surface EEG recordings were in a subset of participants to measure changes in brainwave frequency/activity throughout the test protocol as an objective indicator of each participant's stress level with and without TENS-SLN to assess tolerance of the stimulation protocol. Regardless of type of physiological stress (e.g., tension, anxiety, excitement), the brain's natural response is a decrease in alpha waves (8-13 Hz; indicating a calm and relaxed state) and a corresponding increase in faster brainwaves, particularly beta waves (14-30 Hz; indicating an active, vigilant state) and gamma waves (>30 Hz; indicating a state of hypervigilance and stress). Due to the non-invasive nature of the TENS-SLN procedure, an increase in stress levels did not occur unless the SLNi was inadvertently missed and instead recruited nearby muscles in the anterior neck to cause visible/perceptible off-target muscle contractions necessitating repositioning of the electrodes.

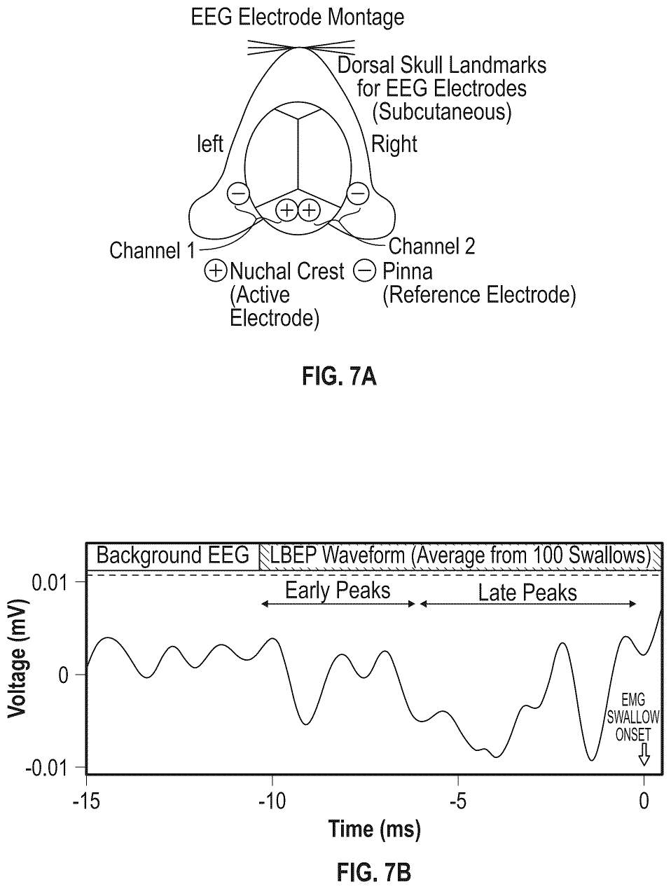

In addition, EEG was used during TENS-SLN to extract laryngeal brainstem evoked potentials (LBEPs) to objectively quantify the electrical activity generated by the neural circuitry during swallowing versus LAR events. This is possible in laboratory rodents (FIGS. 7A-7B), in which repetitively evoked resulted in an averaged LBEP waveform consisting of 8 replicable peaks within 10 ms immediately preceding the onset of EMG swallowing activity. Lesion studies are currently underway to identify the generator source(s) for each peak. In theory, each response peak corresponds to one or more neuroanatomical component in the swallow reflex circuit that is activated in response to SLNi stimulation. Importantly, evoking the LAR in rodents is inconsistent, which results in insufficient LAR events for successful LBEP signal averaging. This barrier was overcome in humans based on the following results that show that the LAR can be reliably evoked repeatedly using air pulse stimulation delivered through the endoscope working channel. See e.g., Lever et al., “Advancing Laryngeal Adductor Reflex Testing Beyond Sensory Threshold Detection” Dysphagia, 2021, which is hereby incorporated by reference in its entirety herein. However, a major advantage of using TENS-SLN (instead of air puffs) to evoke the LAR is that hundreds of responses may be evoked without drying/desensitizing the larynx during testing. Moreover, if successful, the TENS-SLN approach would enable LAR testing to proceed without the need for endoscopy, once the LAR EMG signal is reliably confirmed in this pilot study. Collectively, the preliminary work with laboratory rodents and human subjects provides sufficient feasibility for including non-invasive LBEP testing in this pilot study with healthy adults.

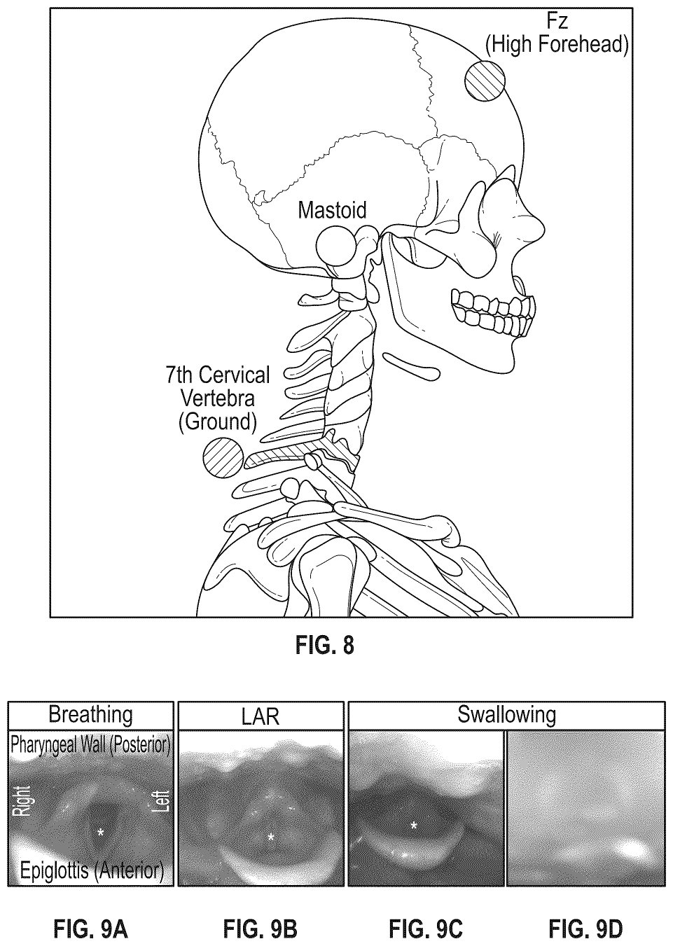

For surface EEG recordings, two electrode pairs were attached to each participant's scalp for synchronous recording of left versus right brainwave activity (FIG. 8), which corresponds with the optimized LBEP electrode configuration in the experimental animal studies described above. It also is a commonly used EEG montage for recording human auditory brainstem evoked potentials. Following testing, the EEG recordings were analyzed offline for quantification of stress level and LBEP activity. Thus, the EEG recording does not add any additional time to the test procedure beyond initial placement of the EEG electrodes. For stress level quantification, the area under the curve was calculated for alpha, beta, and gamma EEG brainwave frequencies for comparison across baseline and TENS-SLN trials.

For LBEP quantification, the tiny evoked responses, which are synchronized to the electrical stimulus, were amplified and extracted from the much larger background EEG signal. Signal-to-noise ratio was improved by signal averaging all of the evoked swallowing versus LAR responses for each participant, which should result in an averaged evoked potential waveform consisting of several positive and negative peaks occurring within milliseconds of stimulation, similar to the above-mentioned findings from laboratory rodents. An averaged LBEP waveform was generated for each TENS-SLN condition (swallowing versus LAR and left versus right side) for objective quantification of the following 2 outcome measurements for each response peak: amplitude (peak-to-peak) and latency from the time-locked EMG reflex (swallowing versus LAR) activity. Response amplitude reflects the number of neurons and degree of synchronized firing within each neural component in the circuit, whereas response latency provides a quantitative indicator for the time course of activation and conduction between neural relays in the circuit.

### Transnasal Endoscopy

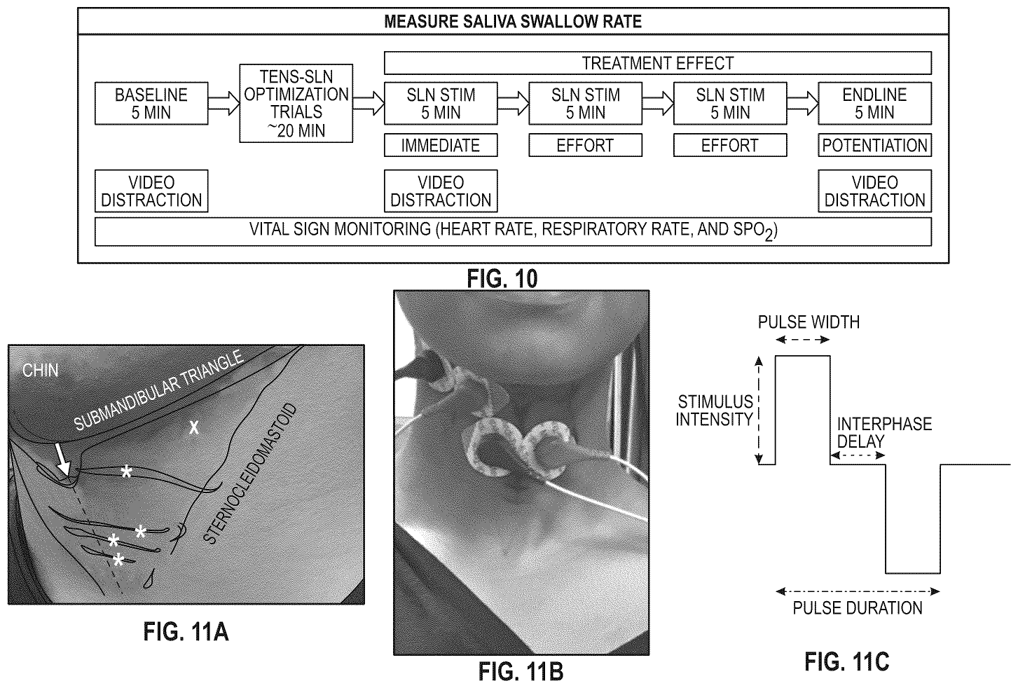

In a subset of up to 20 subjects, transnasal endoscopy was performed in synchrony with TENS-SLN for confirmation of swallowing versus LAR events in the laryngeal EMG recordings. This minimally invasive procedure is essential because EMG activity was relied on to quantify swallow and LAR event rates for this study. Briefly, with the subject seated in an ENT exam chair, a 50:50 mixture of Afrin/2% Lidocaine was administered bilaterally into the participant's nose (targeting only the nasal septum, 0.4 mL per side) and allowed to decongest/anesthetize for 5-10 minutes to enhance patient comfort throughout the transnasal endoscopic procedure. The endoscope was then transnasally advanced through the more patent naris (identified by the participant) and carefully guided passed the soft palate into the pharyngeal space, where it remained just above the tip of the epiglottis for bilateral visualization of the larynx in the camera field of view (FIGS. 9A-9D). Normal/healthy structure/function of the larynx was confirmed by the endoscopist prior to proceeding with the study protocol. In cases of apparent laryngeal mucosal irritation/redness/swelling or left/right asymmetry, the procedure is terminated (i.e., the endoscope is removed, and the TENS-SLN protocol is not proceeded with) and the endoscopist discusses the abnormal findings with the subject. Otherwise, the endoscope remains in this “home position” throughout multiple (up to 12, see Table 3) blocks of TENS-SLN-evoked swallowing versus the LAR, based on individual patient tolerance. The endoscope may be removed between blocks as needed (i.e., for clinician “ergonomic” rest breaks or upon request by the participant) and reinserted for subsequent blocks to permit ample video recording of endoscopic swallowing versus LAR events in synchrony with laryngeal EMG recordings. EMG responses were analyzed relative to the area under the curve, allowed ready identification and distinction between swallowing versus LAR events in the EMG recordings for accurate quantification of event counts/rates. AUC values were higher for swallowing due to oral, pharyngeal, and laryngeal muscle recruitment, compared to relatively smaller AUC values for LAR events because only laryngeal muscles are recruited.

### Infection Control Procedures

The center for disease control guidelines and local hospital system infection control guidelines that were followed are summarized below.

TABLE 3
Infection control procedures

| Procedures | Components | | Level of Cleaning |
|---|---|---|---|
| Ultrasound (non- | Equipment | Ultrasound device | LLD |
| invasive) | | Laptop computer or tablet | LLD |
| | Supplies | Transducer covers | n/a (disposable) |
| Electrophysiology | Equipment | Electrical stimulation device | LLD |
| (non-invasive) | | A/D converter | LLD |
| | | Bioamplifiers | LLD |
| | | Laptop computer | LLD |
| | Supplies | Electrodes | n/a (disposable) |
| | | Sensors (ECG, respiratory, | LLD |
| | | pulse oximetry) | |
| | | Electrode leads | LLD |
| Transnasal | Equipment | Laryngoscope | HLD (central processing) |
| Endoscopy | Supplies | n/a | n/a |
| (minimally | | | |
| invasive) | | | |

LLD = low level decontamination; HLD = high level decontamination.

### Data Safety Monitoring Plan

It is to be appreciated that it can be beneficial to collect data through the related monitoring, audits, IRB/IEC review, and regulatory inspection(s) by providing direct access to source data/documents. This allows research team members, medical professionals, and/or artificial intelligence to analyze all data (e.g., endoscopic videos, electrophysiological recordings, etc.) following each session to ensure ongoing validity and integrity of the research/medical protocol(s). If the data suggests a potential health issue that was not identified during the participant's test session, it can be reported back to the participant, and the participant can be advised to consult their physician. All vital sign data, EMG and EEG data, electronic stimulus data and endoscopic video data can be collected and stored in a de-identified manner. The participants can be assigned an ID number, and this number can be limited to reveal only their age and gender. It can be beneficial, such as to comply with governmental regulations and/or to limit the inadvertent disclosure of sensitive information, that no record of the participant's name or other information were associated with any of the video/data files. The data can be stored electronically on a secure network drive that is both encrypted and password protected and regularly backed up to prevent data loss. Only research personnel and physicians associated with the tests and/or studies have access to this data. Video recordings can be limited such that they do not include the face or any other identifying information. As an added data safety plan, videos may only be viewed only on devices that are intended for these tests; and researchers and medical professions can assure participants that their data will never be viewed on personal devices. These steps can help ensure privacy for the study participants.

### Results

FIG. 10 shows a detailed flowchart of an example TENS-SLN protocol. After obtaining baseline swallowing rate and vital signs, TENS-SLN optimization ensued.

In the main study, example data was collected from healthy adults over twenty years in age, that spoke English as a primary language, were non-smokers, and were otherwise healthy. Participants had to consent to: (1) filling-out questionnaires to assess their health and swallowing function status; (2) laryngeal palpation and/or ultrasound for the placement of electrodes; (3) non-invasive transcutaneous electrical stimulation of the internal branch of the superior laryngeal nerve (TENS-SLN) to evoke swallowing or the laryngeal adductor reflex (LAR); (4) non-invasive electrophysiology recordings (vital signs and EMG for all subjects, and EEG in a subset of subjects); and (5) transnasal endoscopy in a subset of subjects for confirmation of swallowing versus LAR events in the laryngeal EMG recordings. Importantly, the consent process took place in a quiet room with the door closed and minimal personnel present. The participants were encouraged to ask questions and be given ample time to make an informed decision as to their participation. Informed consent was obtained on the day of participation. Other screening procedures included a transnasal endoscopic examination of the nasal cavity, pharynx, and larynx.

The study enrolled 60 healthy adults (˜50:50 males/females) representing 3 age groups in this feasibility and safety study: young adults (20-39 years), middle-aged adults (40-59), and older adults (60 and over). Including these 3 age groups enabled the determination of whether there are any age-related barriers to success of the TENS-SLN protocol in preparation for future clinical trials with patients with dysphagia. From extensive previous swallowing and LAR-based research it is known that 10 subjects per group are suitable for robust statistical analysis. It was important to have equal representation of both sexes for statistical analysis. Therefore, the enrollment of 10 males and 10 females per age group (young, middle, and old) met this criterion.

A first example optimization test to determine optimized electrode placement for TENS-SLN treatment commenced. After identifying the ideal anatomical locations for potential locations for the electrodes, as shown in FIG. 11A, good locations for the electrodes were tested in the positions shown in FIG. 11B. The improved accuracy in placement of electrodes in the carotid triangle where the SLN is situated, along with an increase in the interphase delay from 50 μs to 100 μs as shown in FIG. 11C, were factors that contributed to effective optimization of the TENS-SLN protocol. The biphasic square waveform of FIG. 11C was found to minimize tissue and nerve damage.

In a second example optimization test, and during TENS-SLN treatment (5-minutes), the swallowing rate was increased in Participants 1 and 2 by 2,100% and 1,200%, respectively (i.e., immediate treatment effect). Both participants demonstrated a potentiation (carry-over) effect of 900% during the 5-minute period following TENS-SLN treatment. In both subjects, swallowing was evoked on the first trial, indicating success in optimizing the TENS-SLN protocol.

TABLE 4
TENS-SLN outcomes

| | Optimization | | Stim Trial Swallow # | | | Endline Swallow # | |
|---|---|---|---|---|---|---|---|
| Participant | # Sessions | # Trials | Baseline | SLN Stim | % change | Endline | % change |
| 1 (Female) | 1 | 1 | 1 | 22 | 2100% | 10 | 900% |
| 2 (Female) | 1 | 1 | 1 | 13 | 1200% | 10 | 900% |
| 1 (Female) | 2 | 7 | 1 | 38 | 3700% | 5 | 400% |
| 2 (Female) | 1 | 2 | 4 | 16 | 1500% | 7 | 75% |

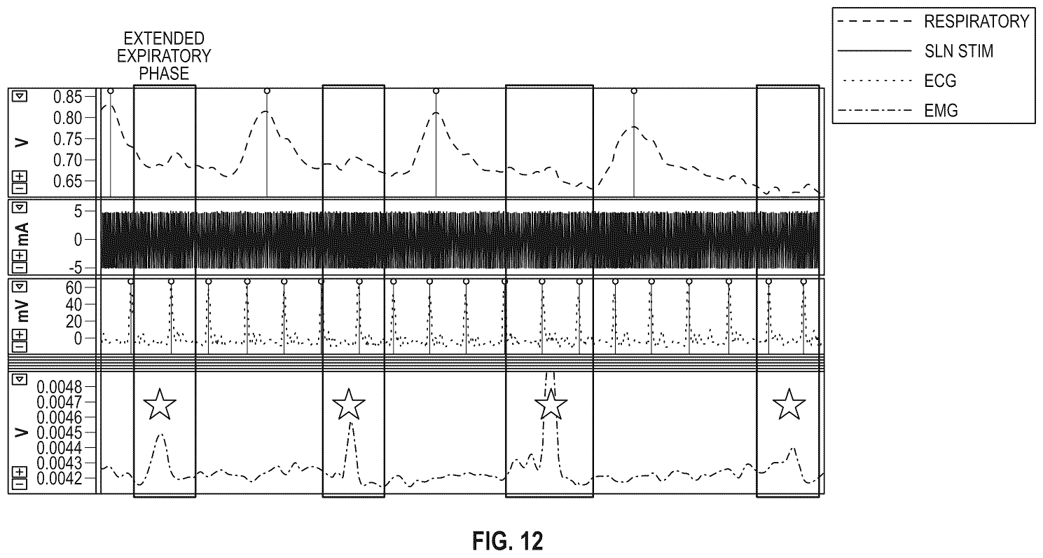

FIG. 12 shows that multiple stimulus intensities (between 5-10 mA) were tested to determine the therapeutic level that elicited 2-4 swallows per 20 second stimulus train, identified by laryngeal EMG peaks (red stars) in synchrony with laryngeal elevation (via webcam recording) and disrupted respiratory pattern (i.e., brief apnea). Following optimization, 3 TENS-SLN trials (5-minutes each) were conducted, separated by a brief (˜1-minute) break to ensure participant tolerance and electrode adherence. For the 2^{nd} and 3^{rd} TENS-SLN trials, the participant was instructed to forcibly squeeze the swallowing muscles (i.e., effortful swallow maneuver) during every other evoked swallow. Before each 5-minute trial started, each participant swallowed 3 mL of bottled water (room temperature), self-dispensed from a medicine cup. Vital signs (respiratory rate, heart rate, and SpO₂) were captured in synchrony with SLN stimulation and laryngeal EMG recordings.

The TENS-SLN protocol reliably evoked repetitive swallowing in healthy participants, as shown in Table 5.

| | Optimization | | # Swallows | | | | |
|---|---|---|---|---|---|---|---|
| | # | | | SLN | % | # Swallows | % |
| Participant | Sessions | # Trials | BASELINE | Stim | Change | ENDLINE | Change |
| 1 (Female) | 1 | 1 | 13 | 33 | 154 | 10 | −23 |
| 2 (Female) | 3 | 25 | 2 | 23 | 1050 | 1 | −50 |
| 3 (Male) | 2 | 7 | 1 | 17 | 1600 | 1 | 0 |
| 4 (Male) | 5 | 40 | 4 | 14 | 250 | 2 | −50 |
| 5 (Female) | 2 | 7 | 1 | 38 | 3700 | 5 | 400 |
| 6 (Male) | 1 | 2 | 8 | 29 | 263 | 16 | 100 |
| 7 (Female) | 1 | 2 | 4 | 16 | 300 | 7 | 75 |
| 8 (Female) | 1 | 1 | 5 | 30 | 500 | 9 | 80 |
| 9 (Male) | 1 | 1 | 3 | 25 | 733 | 3 | 0 |
| | | | | | Immediate Effect | | Potentiation |

TENS-SLN reliably evoked repetitive swallowing in 9/9 (100%) participants; however, 4/9 (44%) required 2-5 sessions for success. The immediate treatment effect ranged from a 154-3,700% increase over baseline swallow rate. Only 2 of the participants demonstrated a potentiation (carry-over) effect during the 5 minutes following TENS-SLN. This finding suggests that further optimization could be beneficial.

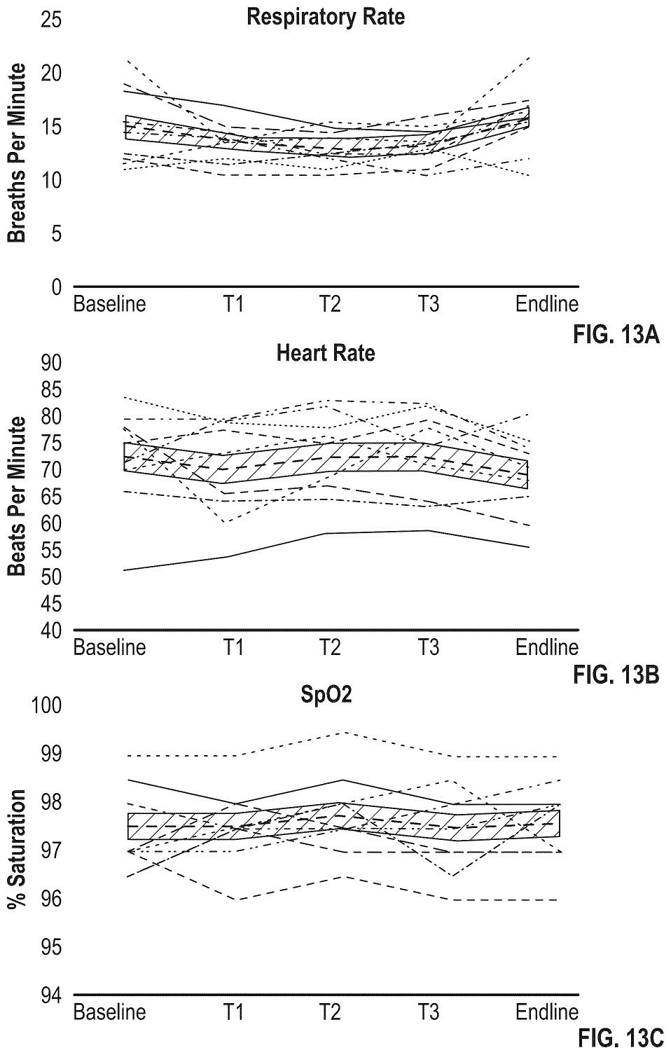

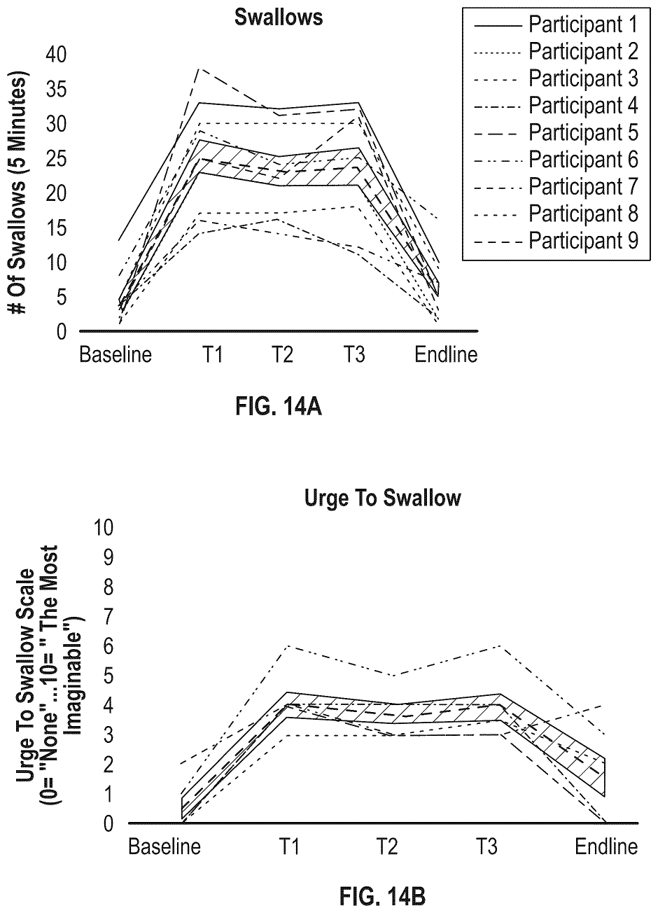

TENS-SLN does not have any adverse effects on vital signs in humans, as shown in FIGS. 13A-13C. TENS-SLN increases swallow rate, without fatigue, and the “urge to swallow”, as shown in FIGS. 14A-14B.

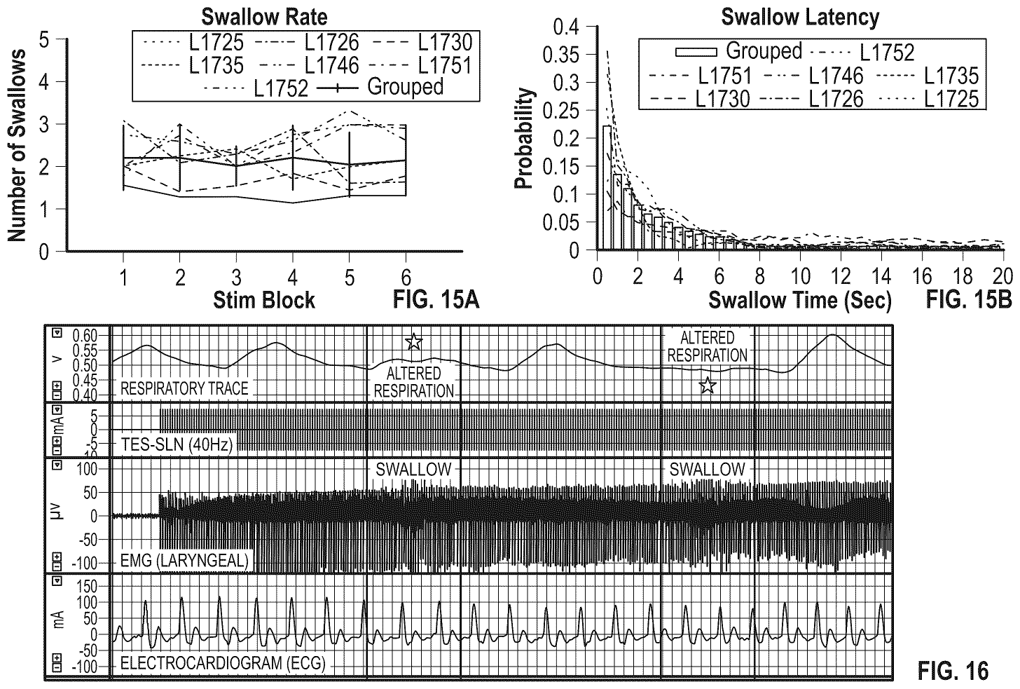

Clinical results with mice showed surgically applied electrical stimulation of the superior laryngeal nerve reliably evoked swallowing throughout a 30-minute treatment session (FIGS. 15A-15B). Moreover, a single 30-minute SLN stimulation treatment at 6 months of age remarkably maintained swallowing function as the disease advanced and had no adverse effects on respiratory rate throughout the procedure.

Referring back to the main study, FIG. 16 shows LabChart software was used to record respiration, TENS-SLN, laryngeal EMG, and heart rate. SpO2 was recorded manually at the beginning and end of each session. The results show that swallowing alters the respiratory trace (red star indicating brief apnea events). Swallows were confirmed by EMG bursting in synchrony with laryngeal elevation, as evidenced by a synchronized video recording that coincided with altered respiratory trace.

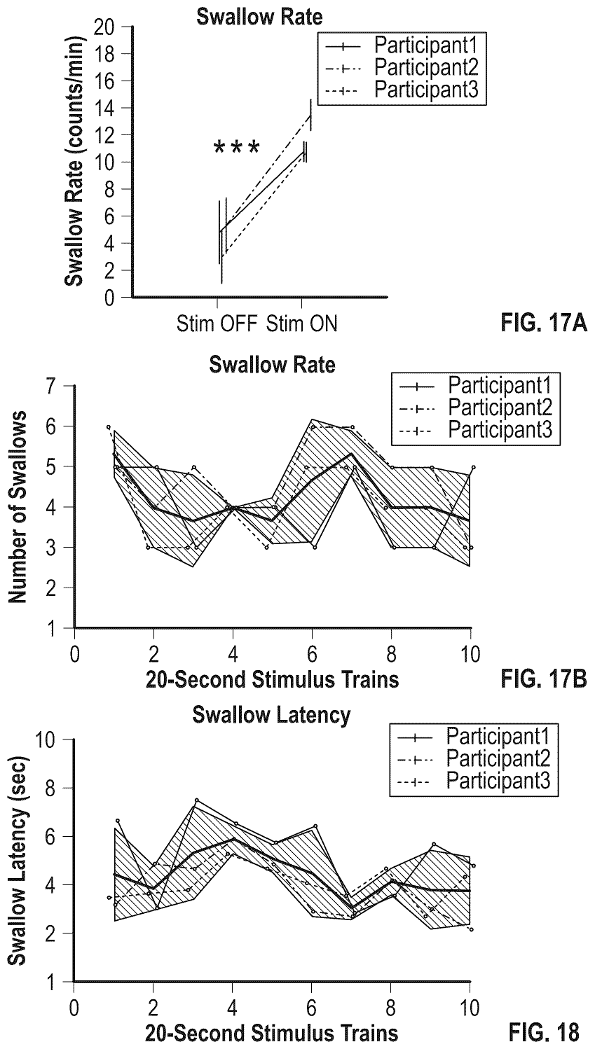

FIG. 17A shows TENS-SLN significantly increased saliva swallow rate compared to baseline (p<0.0001, ***). FIG. 17B shows swallow rates were compared between each 20-second stimulation train throughout a single 5-minute treatment session, which revealed no significant differences (p=0.58; ANOVA); black line=mean; gray shading=SEM.

FIG. 18 shows swallow latencies were compared between each 20-second stimulation train throughout a single 5-minute treatment session, which revealed no significant differences (p=0.98; ANOVA); black line=mean; gray shading=SEM.

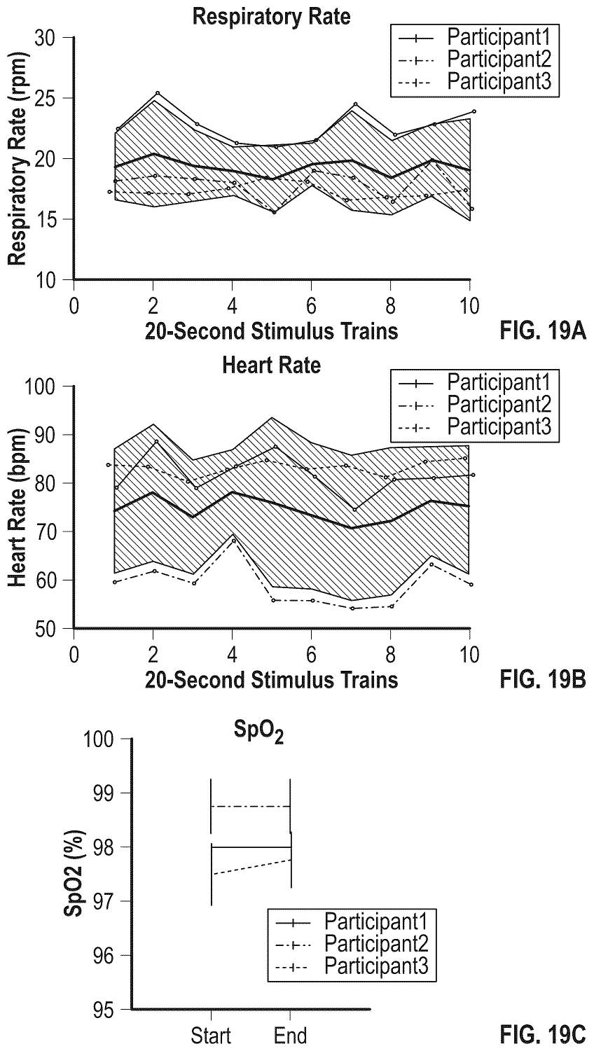

FIGS. 19A-19B compare respiratory rate (FIG. 19A) and heart rate (FIG. 19B) between each 20-second stimulation train throughout a single 5-minute treatment session, which revealed no significant differences (p=0.88 and p=0.76, respectively; ANOVA). FIG. 19C shows there was no significant difference (p=0.58; ANOVA) in SpO₂ from the Block Start to Block End. Black line=mean; gray shading=SEM.

From the foregoing, it can be seen that the present disclosure accomplishes at least all of the stated objectives.

### Glossary

Unless defined otherwise, all technical and scientific terms used above have the same meaning as commonly understood by one of ordinary skill in the art to which embodiments of the present disclosure pertain.

The terms “a,” “an,” and “the” include both singular and plural referents.

The term “or” is synonymous with “and/or” and means any one member or combination of members of a particular list.

As used herein, the term “exemplary” refers to an example, an instance, or an illustration, and does not indicate a most preferred embodiment unless otherwise stated.

The term “about” as used herein refers to slight variations in numerical quantities with respect to any quantifiable variable. Inadvertent error can occur, for example, through use of typical measuring techniques or equipment or from differences in the manufacture, source, or purity of components.

The term “substantially” refers to a great or significant extent. “Substantially” can thus refer to a plurality, majority, and/or a supermajority of said quantifiable variables, given proper context.

The term “generally” encompasses both of the terms “about” and “substantially.”

The term “configured” describes structure capable of performing a task or adopting a particular configuration. The term “configured” can be used interchangeably with other similar phrases, such as constructed, arranged, adapted, manufactured, and the like.

Terms characterizing sequential order (e.g., first, second, third), a position, and/or an orientation are not limiting and are only referenced according to the views presented.

When an element is referred to as being “connected,” “coupled,” “mated,” “attached,” “fixed,” etc. to another element, the element can be directly connected to the other element, or intervening elements may be present. In contrast, when an element is referred to as being “directly connected,” “directly coupled,” etc. to another element, there are no intervening elements present. Other words used to describe the relationship between elements should be interpreted in a like fashion (e.g., “between” versus “directly between,” “adjacent” versus “directly adjacent,” etc.). Similarly, terms such as (i) “communicatively connected” or (ii) “fluidly connected” include (i) all variations of information exchange and routing between two electronic devices, including intermediary devices, networks, etc., connected wirelessly or not and (ii) all variations of fluid exchange and routing between two fluidic bodies, including intermediary fluid paths, flows, etc., connected indirectly or not.

The “invention” is not intended to refer to any single embodiment of the particular invention but encompass all possible embodiments as described in the specification and the claims. The “scope” of the present disclosure is defined by the appended claims, along with the full scope of equivalents to which such claims are entitled. The scope of the disclosure is further qualified as including any possible modification to any of the aspects and/or embodiments disclosed herein which would result in other embodiments, combinations, subcombinations, or the like that would be obvious to those skilled in the art.

What is claimed is:

1. A method of evoking swallowing or the laryngeal adductor reflex (LAR) comprising:
applying a pair of surface electrodes at a specific location on an anterior neck; and
delivering an electrical code a brainstem region via the superior laryngeal nerve (SLN) branch of the vagus nerve to evoke either swallowing or the LAR.

2. The method of claim 1, wherein the LAR is a code-dependent response.

3. The method of claim 1, further comprising targeting an internal branch of the SLN (SLNi) and upstream brainstem synapses to reliably evoke swallowing rather than merely causing local muscle contractions, wherein the swallowing is a code-dependent response.

4. The method of claim 3, further comprising moving the stimulating electrodes laterally over the larynx and to selectively stimulate the SLNi.

5. The method of claim 3, further comprising positioning the stimulating electrodes parallel with the SLNi to drive an electrical current flow toward the brainstem and away from the muscles in the neck.

6. The method of claim 1, wherein the method is free from using surface electrodes to stimulate and strengthen muscles in the neck without evoking an urge to swallow.

7. A therapeutic method comprising the method of claim 1, further comprising: utilizing TENS-SLN to selectively treat multiple airway protective reflexes.

8. A diagnostic method comprising the method of claim 1, further comprising diagnosing a health condition based on detection of a neural response to the delivery of the precise electrical code to the corresponding region of the brainstem via the SLN.

9. The diagnostic method of claim 8, further comprising accomplishing said detection utilizing surface laryngeal electromyography.

10. The diagnostic method of claim 8, further comprising accomplishing said detection utilizing transnasal endoscopy.

11. The diagnostic method of claim 7, further comprising accomplishing said detection utilizing respiratory plethysmography.

12. The diagnostic method of claim 7, further comprising accomplishing said detection utilizing laryngeal palpation.

13. The diagnostic method of claim 7, wherein the diagnosis comprises dysphagia due to a condition selected from the group consisting of: (i) Parkinson's disease; (ii) stroke; (iii) amyotrophic lateral sclerosis; and (iv) cancer.

14. A handheld transcutaneous electrical nerve stimulation (TENS)/neuromuscular electrical stimulation (NMES) unit that evokes swallowing or the laryngeal adductor reflex comprising:
a portable body;
a display;
an electrical stimulator including:
a pair of electrode leads that can be attached to surface electrodes that can be attached to a mammalian body;
a transmitter that allows for pulsing an electrical code to a location of a brainstem region of the mammalian body;
wherein a location of the electrodes is over the superior laryngeal nerve (SLN), and the reflex that is evoked depends on information within the electrical code.

15. The handheld TENS/NMES unit of claim 14, wherein the handheld TENS/NMES unit comprises a transcutaneous electrical nerve stimulation superior laryngeal nerve (TENS-SLN) unit.

16. The handheld TENS/NMES unit of claim 14, further comprising a user interface that allows the operator to input a stimulus frequency between 0.1 and 100 Hz.

17. The handheld TENS/NMES unit of claim 14, further comprising a user interface that allows the operator to input a pulse width and interphase delay between ten microseconds (10 μs) and one millisecond (1 ms).

18. The handheld TENS/NMES unit of claim 14, further comprising a user interface that allows the operator to input a stimulus polarity selected from the group consisting of: (i) anodal and/or toward the brain; and (ii) cathodal and/or toward the muscles.

19. The handheld TENS/NMES unit of claim 14, further comprising a user interface that allows the operator to input a length of a duration and/or a pattern of the pulsing.

20. The handheld TENS/NMES unit of claim 14, wherein the display is configured to output participant data selected from the group consisting of: (a) sensory threshold intensity (mA); (b) therapeutic stimulus intensity (mA); (c) change in swallow frequency (swallows per minute); (d) swallow latency; (e) respiratory rate; (f) heart rate; and (g) blood oxygen saturation, from baseline values.
